(19) United States
(12) Patent Application Publication
(10) Pub. No.: US 2020/0377855 A1
(43) Pub. Date: Dec. 3, 2020

(54) RAPID METHOD FOR THE CULTURE OF TUMOR INFILTRATING LYMPHOCYTES

(71) Applicant: H. LEE MOFFITT CANCER CENTER AND RESEARCH INSTITUTE, INC., Tampa, FL (US)

(72) Inventors: Linda L. Kelley, Tampa, FL (US); Nermin Gerges, Orlando, FL (US); John Ellis Mullinax, Tampa, FL (US); Shari Pilon-Thomas, Tampa, FL (US); Amod Ashok Sarniak, Tampa, FL (US); MacLean Scott Hall, Tampa, FL (US)

(21) Appl. No.: 16/610,681

(22) PCT Filed: May 4, 2018
(86) PCT No.: PCT/US2018/031050

§ 371 Date: Nov. 4, 2019

Related U.S. Application Data
(60) Provisional application No. 62/501,977, filed on May 5, 2017.
(60) Provisional application No. 62/571,969, filed on Oct. 13, 2017.
(60) Provisional application No. 62/612,915, filed on Jan. 2, 2018.

(51) Int. Cl. C12N 5/0783; A61K 35/17

(52) U.S. Cl. CPC C12N 5/0636; C12N 2501/25; C12N 2501/2302; A61K 35/17

(57) ABSTRACT

Disclosed are methods for rapidly expanding tumor infiltrating lymphocytes.

### I. BACKGROUND

Tumor infiltrating lymphocytes (TILs) are mononuclear cells that have left the bloodstream and migrated into a tumor. TILs have been used in autologous adaptive transfer therapy for the treatment of cancer. Typically, a fresh surgically resected tumor is used as the starting material for successful initiation and expansion of tumor specific TIL culture to manufacture a clinically relevant dose of TIL therapy. Therefore, the candidate patient for TIL therapy needs to be eligible for surgery. If the patient is eligible for surgery, the tumor needs to be resectable. If several tumor anatomical sites are present, a skilled choice of resection of the suitable tumor met(s) with potential T cell infiltration must be made for each patient.

In the production of TILs, once a surgically resectable tumor has been obtained, the tumor is typically cut into small fragments and multiple fragments placed into wells of a culture plate where initial TIL expansion (referred to as “Pre-REP”) occurs. The initially expanded TIL population is then subject for a second round of expansion (referred to as “REP”) in tissue culture flasks. In total, 5-7 weeks of culture are needed and the culture conditions necessitate the use of a cleanroom, splitting of cultures to check confluence, and considerable time to maintain the cells. What are needed are novel methods of rapidly expanding TILs that are less invasive, faster, require less processing, and require less resources to expand the culture.

### II. SUMMARY

Disclosed are methods and compositions related to rapidly producing an expanded tumor infiltrating lymphocyte (TIL) population.

In one aspect, disclosed herein are methods of rapidly producing an expanded tumor infiltrating lymphocyte (TIL) population comprising a) plating TILs obtained from a tissue sample in a gas permeable reservoir (such as, for example, a GREX24 plate); b) culturing the TILs in media (such as for example complete media) comprising IL-2; and c) exchanging the media in the reservoir at least 2 times per week.

In one aspect, the TILs are obtained from one or more core biopsy tissue samples. Also disclosed herein are methods of any preceding aspect, wherein the one or more core biopsies are plated directly from the patient without disaggregation of the specimen. In one aspect, disclosed herein are methods of any preceding aspect wherein each core biopsy is separately cultured in a gas permeable reservoir (such as, for example, a single core biopsy in a single well of a GREX24 plate). Also disclosed are methods of any preceding aspect, further comprising performing one or more core biopsies before plating the TILs. In one aspect, also disclosed herein are methods of rapidly producing an expanded TIL population further comprising harvesting the expanded TIL population.

In one aspect, disclosed herein are methods of any preceding aspect wherein the complete media further comprises anti-41BB (anti-CD137) antibody.

Also disclosed are methods of any preceding aspect, wherein the gas permeable reservoir is a gas permeable tissue culture multi-well plate (such as, for example, a GREX24 plate).

The disclosed expanded TIL population can be used for the treatment of cancer. In one aspect, disclosed herein are methods of treating a cancer in a subject comprising administering to the subject the rapidly expanded TIL of any preceding aspect. In other words, disclosed herein, in one aspect, are methods of treating cancer in a subject comprising obtaining one or more core biopsies comprising TIL from the subject; plating the one or more core biopsies comprising TILs each in a gas permeable reservoir; culturing the cells from the biopsy in a complete media comprising IL-2 for 5 weeks or less; harvesting the expanded TIL cells; adoptively transferring to the subject the expanded TIL.

### III. BRIEF DESCRIPTION OF THE DRAWINGS

FIG. 1 shows the maximum TIL count obtained per well of a G-REX 24-well culture plate is sufficient as our requirement for viable TIL count per cultured tumor fragment for TIL clinical dose production. The produced TIL viability.

FIG. 2 shows the TIL expansion in G-REX 24-well plates was sustained when media and IL-2 replenishment was maintained at 3 times per week.

FIGS. 3A, 3B, 3C, 3D, 3E, and 3F show that culture in GREX24 plates significantly increased viability (3A and 3B), yield (3C and 3D), and percent viability after expansion (3E and 3F) relative to conventional 24-well plates when cultured with IL-2 or IL-2 and 4-1BBL for 21 or 28 days.

FIG. 4 shows the protocol for core biopsy pre-REP.

FIG. 5 shows that the improved methodology of a single core biopsy in a single well resulted in better yield than multiple fragments in a single well even when both fragments and core biopsies were grown in GREX24 plates.

FIGS. 6A and 6B show that the protocol of FIG. 4 produces sufficient TIL yield 7 days faster in GREX24 plates than traditional plates and was not improved by further culture (6A), but further culture to show a reduction in viability (6B).

FIG. 7 shows that the increased viability in GREX24 plates using core biopsies and the new methodology compared to conventional methodology (i.e., surgical resection followed by fragment production, digest, and culture of multiple fragments in a single well of a regular 24-well plate) was maintained when culturing dedifferentiated liposarcoma, undifferentiated pleomorphic sarcoma, and desmoplastic small round cell tumors.

FIGS. 8A, 8B and 8C show the immune phenotype of TILs using the core biopsy protocol and culturing in GREX24 plates.

FIG. 9 shows the TIL phenotype differences between the culture methods for different tumor types.

FIG. 10 shows that the reactivity against autologous tumor increased using the improved core biopsy TIL culture method relative to prior methodology.

FIG. 11 shows the TIL yield per bladder cancer cores receiving IL-2 alone or IL-2 and anti-4-1BB.

FIG. 12 shows the total amount of T cells and the percentage of CD4+ T cells and CD8+ T cells when cultured in IL-2 alone or both IL-2 and anti-4-1BB.

FIG. 13 shows the IFN-g secretion by TILs from bladder cancer cores following culture with IL-2 or co-culture of IL-2 and anti-4-1BB.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Tumor infiltrating lymphocytes (TILs) are mononuclear cells that have left the bloodstream and migrated into a tumor. TILs have been used in autologous adaptive transfer therapy for the treatment of cancer. Typically, a fresh surgically resected tumor is used as the starting material for successful initiation and expansion of tumor specific TIL culture to manufacture a clinically relevant dose of TIL therapy. Therefore, the candidate patient for TIL therapy needs to be eligible for surgery. If the patient is eligible for surgery, the tumor needs to be resectable. If several tumor anatomical sites are present, a skilled choice of resection of the suitable tumor sites with potential T cell infiltration must be made for each patient.

Before TIL production can begin in the prior art methods, a surgically resectable tumor must be obtained. The acquisition of tumor for TIL culture (first step in TIL therapy, called preREP) requires a surgical procedure. The surgical resection is very invasive and could be dramatically simplified if core biopsies could be used. Nevertheless, the invasive acquisition of a tissue sample is not the end of the problems for the protocols in use prior to the present disclosure. After acquisition, the tissue samples from prior methods must go through intensive laboratory preparation of the tumor for culture. In fact, 5-7 weeks of culture are needed and the culture conditions necessitate the use of a cleanroom, splitting of cultures to check confluence, and considerable time to maintain the cells.

The production method of TILs for TIL therapy would be significantly less invasive if biopsy specimens, such as core biopsies, could be used rather than a tumor resection which is further divided and subject to processing (i.e., digest) before culturing. Nevertheless, prior attempts to use biopsy specimens have not been successful and it is commonly understood to be a non-reliable method of initiating a TIL culture. Sarnaik et al. (US Patent Application Publication No. US2017/0044496) attempted to optimize preREP culture through the addition of anti-4-1BB to the culture to increase yield and CD8+ fractions. However, the methods still required surgical tumor removal, dividing the sample into fragments, and processing the sample fragments in the laboratory into digest which was used to initiate TIL culture. Rosenberg et al. (US Patent Application Publication No. US2017/0152478) cultured multiple processed fragments for a surgical resected tumor in GREX 10 flasks in an attempt to increase TIL yield and decrease pre-REP culture, but never assessed tumor reactivity of the TIL culture and ignores the tumor heterogeneity by having multiple fragments in a single culture. Disclosed herein are less invasive more rapid methods of expanding TILs.

To overcome the obstacles of the methods employed in the art, Applicants developed a reliable method to initiate TIL culture from percutaneous tumor samples with no preparatory work required. Moreover, the method disclosed herein recognizes that individual fragments of tumor yield dramatically different TIL cultures with different degrees of efficacy against tumor. The method requires no laboratory preparation of specimen between patient and incubator (decreased cost and time) and eliminates need for surgical resection (decreased cost both financial and risk), These advantages increase eligibility for treatment with TIL (allows accrual of unresectable patients). Additionally, by using core biopsies rather than a surgically resected tumor, the method allows for the image guided sampling of high yield regions in heterogeneous tumors (i.e. viable regions rather than necrosis).

In one aspect, disclosed herein are methods of rapidly producing an expanded tumor infiltrating lymphocyte (TIL) population comprising a) plating TILs obtained from one or more tissue samples in a gas permeable reservoir (such as, for example, a GREX24 plate); b) culturing the TILs in complete media comprising IL-2; and c) exchanging the media in the reservoir on a regular basis until harvest.

The novel method of growing TIL provided herein is significantly more effective in culturing TILs, compared to the current art accepted method. As noted above, to obtain TILs, the current methods employed in the art utilize surgically removed tissue sections obtained from tumors, and therefore rely on the tumor itself being resectable with acceptable morbidity to the patient. By contrast, the disclosed methods can utilize the initiation and expansion of TIL from biopsies (such as, for example, core-biopsies including core needle biopsies) rather than surgically resected tumors. This represents a novelty compared to the current art utilized TIL manufacturing process.

In one aspect, the disclosed methods of producing an expanded TIL population comprise obtaining a biopsy from the subject (such as, for example, percutaneous tumor samples). As used herein, “biopsy” can include any partial removal of a tissue such as excisional, incisional, core, or fine needle aspiration biopsies. In one aspect, it is understood that core biopsies (including core needle biopsies) are preferred as incisional and excisional biopsies require an operation and thus pose no advantage of surgical resection. By contrast core biopsies (including core needle biopsies) can be performed via percutaneous tumor sampling. Due to the quantities of material obtained, core biopsies are also preferred to fine needle aspiration. Thus, in one aspect disclosed herein are methods of producing an expanded TIL population comprising obtaining a biopsy from the subject wherein the biopsy is a core needle biopsy. In one aspect, the core biopsy (including core needle biopsy) is a percutaneous tumor sample. Also disclosed herein are methods of producing an expanded TIL population comprising obtaining a biopsy from the subject wherein the biopsy is not a fine needle biopsy.

It is understood and herein contemplated that the use of TILs obtained from biopsies (such as, for example, core biopsies including core needle biopsies) makes TIL therapy available to patients who are not eligible for surgery and for patients with unresectable tumors. In addition, core biopsies (such as, for example core needle biopsies) allows for image guided sampling from several anatomical sites. As samples from multiple sites can be obtained, the efficacy tumor specific TIL culture is increased. Utilizing TILs obtained from core biopsy (such as, for example core needle biopsies) also results in a decreased risk to the patients due to surgical complications and a significant decrease in the cost of the TIL therapy. Thus, in one aspect, disclosed herein are methods of rapidly producing an expanded tumor infiltrating lymphocyte (TIL) population comprising a) plating TIL obtained from a tissue sample in a gas permeable reservoir (such as, for example, a GREX24 plate); b) culturing the TIL in complete media comprising IL-2; and c) exchanging the media in the reservoir; wherein the TILs are obtained from core biopsy (such as, for example core needle biopsies) tissue sample. It is understood and herein contemplated that the due to the use of core biopsies (such as, for example core needle biopsies) to obtain TILs the method can employ, in one aspect, a step of obtaining TILs by performing a core biopsy (such as, for example core needle biopsies) on the subject. Thus, in one aspect, disclosed herein are methods of rapidly producing an expanded tumor infiltrating lymphocyte (TIL) population comprising a) plating TIL obtained from one or more core biopsy (such as, for example core needle biopsies) tissue samples from obtained from one or more anatomical sites in a gas permeable reservoir (such as, for example GREX 24 plates); b) culturing the TILs in complete media comprising IL-2; and c) exchanging the media in the reservoir; further comprising performing a core biopsy (such as, for example core needle biopsies) before plating the TILs.

Core biopsies (such as, for example core needle biopsies) can be obtained using any device with which a core biopsy can be obtained (see, for example, the Bard Core Biopsy Instruments and Temno Biopsy Systems by Carefusion such as, BARD MAGNUM®, BARD MAX-CORE®, BARD BIOPTY-CUT®, BARD MARQUEE®, BARD MISSION®, and BARD MONOPTY® from CR Bard, Inc.). The needle for obtaining the biopsy can be 6, 8, 10, 12, 14, 16, 18, or 20 gauge needle with a needle length between about 2 cm and to about 30 cm long, preferably between about 10 cm and about 25 cm long, more preferably between about 16 cm and about 20 cm long. For example, the needle length for obtaining a core biopsy can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm long. The penetration depth of the needle can be between about 15 mm and 30 mm, preferably between about 20 mm and 25 mm. For example, the penetration depth can be 15, 16, 17, 18, 19, 20, 21, 22, 23, 24, 25, 26, 27, 28, 29, or 30 mm.

It is contemplated herein that TILs may not be present in every tissue sample (such as, for example, a core biopsy including core needle biopsies). One advantage of the disclosed methods is the use of core biopsies (such as, for example core needle biopsies) is minimally invasive and allows the physician to obtain one or more biopsy tissue samples from one or more anatomical sites or different sites within the same tumor and to accomplish this using image guided sampling). For example, the physician can obtain, 2, 3, 4, 5, 6, 7, 8, 9, 10, 11, 12, 13, 1 4, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 core biopsy samples, from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 anatomical sites. It is understood and herein contemplated that the core biopsies can be utilized directly from the patient without disaggregation of the specimen. Accordingly, in one aspect, disclosed herein are methods of rapidly producing an expanded TIL population wherein the core biopsy (such as, for example core needle biopsies) is plated directly from the patient without disaggregation of the specimen.

In one aspect, the use of core biopsy allows the ability to target certain and possibly multiple areas of a tumor. In one aspect, disclosed herein are methods of rapidly producing an expanded TIL population further comprising the use of imaging techniques such as radiomics to guide TIL acquisition.

Once obtained, tissue core biopsies (such as, for example, core needle biopsies) provided the added advantage of not requiring further sectioning (i.e., making fragments) or processing (for example, digest), but can be placed immediately in culture. In one aspect, the disclosed methods can comprise placing the tissue sample directly into the gas permeable reservoir. In one aspect, disclosed herein are methods of rapidly producing an expanded TIL population wherein the core biopsy (such as, for example, a core needle biopsy) wherein the core biopsy is not subject to further section and/or further processing (for example, digest).

It is additionally recognized herein that the use of multiple fragments in a single culture ignores tumor heterogeneity. Moreover, individual fragments have dramatically different resultant TIL cultures and degree of tumor reactivity. Thus, in one aspect disclosed herein are methods of rapidly producing an expanded TIL population comprising plating TILS obtained from atissue sample in a gas permeable reservoir (such as, for example, a GREX 24 plate); culturing the TILs in media comprising IL-2; and exchanging the media in the reservoir at least 2 times per week; wherein each well of the GREX24 plate contains a single core biopsy (such as, for example, a core needle biopsy).

The culture process employed by the art understood methods takes 5-7 weeks to expand TILs. This is a significant problem in the art as additional time to initiating adoptive transfer therapy of TILs represents an increased risk to the patient due to progression of malignancy while the cell product is being prepared. Moreover, the added time needed for culturing requires additional resources of the hospital in additional personnel to requirements to maintain the culture and costs for media and maintaining a cleanroom. The present method decreases the expansion time to less than 5 weeks resulting in decreased attrition patients from therapy secondary to disease progression. For example, culturing to obtain an expanded population of TILs can occur for any time between 1 day and 5 weeks (35 days), preferably between 21 days (3 weeks) and 5 weeks (35 days), more preferably between 4 weeks (28 days) and 5 weeks (35 days). For example, the culture time can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In one aspect, disclosed herein are methods of rapidly producing an expanded tumor infiltrating lymphocyte (TIL) population comprising a) plating TIL obtained from a tissue sample in a gas permeable reservoir (such as, for example, a GREX 24 plate); b) culturing the TIL in complete media comprising IL-2 for 5 weeks or less; and c) exchanging the media in the reservoir.

To maintain the quality of the nutrients in culture and remove any waste, it is understood and herein contemplated that the all or a portion of the media in the reservoir maybe exchanged. The exchange of media can comprise 5, 10, 15, 20, 25, 30, 35, 40, 45, 50, 55, 60, 65, 70, 75, 80, 85, 90, 95, or 100% removal and replacement of media. This media exchange can be accomplished employing any acceptable method for proper tissue culture maintenance known in the art. In one aspect, the media exchange can occur at least one time during the culture of the TILs. For example, the media in the reservoir can be exchanged 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 times during the culture period. That is, the media exchange can occur once during the culture period, once every 15 days, once every 10 days, once every 7 days, once every 5 days, once every other day, or about 2 to 3 times per week. For example, in one aspect, disclosed herein are methods of rapidly producing an expanded tumor infiltrating lymphocyte (TIL) population comprising a) plating TIL obtained from a tissue sample in a gas permeable reservoir; b) culturing the TIL in complete media comprising IL-2 for 5 weeks or less; and c) exchanging the media in the reservoir at least 2 times per week.

The culture methods employed herein can utilize any complete media comprising IL-2 appropriate for the growth and propagation of the TILs, including, but not limited to Minimum Essential Medium (MEM), Eagles's Minimum Essential Medium (EMEM), Dulbecco's Minimum Essential Medium (DMEM) Medium 199, RPMI 1640, CMRL-1066, BGJb Medium, Iscove's Modified Dulbecco's Medium (IMDM), and Blood Cell Media. In one aspect, the media can comprise anti-41BB (anti-CD137) antibody.

The TILs can be cultured in any gas permeable reservoir suitable for cell culture and the expansion of TILs. In one aspect, it is understood and herein contemplated that large tissue culture flasks can slow down the expansion of TILs as it takes longer for cells to reach confluency. In one aspect, the gas permeable reservoir can be a tissue culture plate comprising 6 (approximately 10 cm² surface area per well and 60 cm² total surface area), 12(approximately 4 cm² surface area per well and approximately 48 cm² total surface area), 24 (approximately 2 cm² surface area per well and approximately 48 cm² total surface area), 48(approximately 1 cm² surface area per well and approximately 48 cm² total surface area), or 96 (approximately 0.32 cm² surface area per well and 31 cm² total surface area) wells (for example, G-Rex24 well plate or G-Rex6 well plate manufactured by Wilson Wolf). In some aspect, the plates can be silicone coated. Such multi-well tissue culture plates allows for the ability for each well to comprise a single core biopsy (such as, for example, a core needle biopsy). Thus, where a 6, 12, 24, 48, or 96 well plate is used, there can be 6, 12, 24, 48, or 96 core biopsy (such as, for example, a core needle biopsy) cultures, respectively in each plate. In other words, each core biopsy (such as, for example, a core needle biopsy) can be cultured and expanded in its own well.

It is understood and herein contemplated that the gas permeable reservoir may also comprise a polyolefin bag (such as, for example, the Charter Medical culture bag EXP-50®). The advantage of such a culture reservoir is that the polyolefin bag comprises a closed system culture bags, which allows the TIL manufacturing process to be in a closed system which in concept requires only a hood and not a cleanroom for manufacturing.

One advantage of the disclosed methods is the ability to take a sample of TILs from each biopsy for testing for numbers, viability, and tumor specificity without disturbing or risk the viability of the entire culture. It is understood and herein contemplated that the disclosed methods improve viability count, TIL yield, or percent yield after expansion relative to traditional methods. In one aspect, the TIL yield, viability and/or percent yield is at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 times greater than the yield obtained via traditional methods.

As the intent of the methods for rapidly producing an expanded TIL population is to use the TILs in adoptive transfer therapy for cancer. The new method results in several advantages from the prior process. First, there is a more successful expansion of TILs from tumor subtypes with previously poor growth. Second, the method offers significantly decreased morbidity compared to open surgical resection. Third, TIL harvest will now be available to patients with tumors that are unresectable due to excessive surgical morbidity. Finally, using image guidance, it is now possible to target specific regions of tumors for TIL harvest. Taken together, this method provides for the successful manufacture of TILs for ACT, at lower risk and decreased cost, to patients that would not have been previously available through the current method. The new method is performed with significantly less technical intervention time resulting in an increase in TIL production efficiency.

Accordingly, disclosed herein are methods of rapidly producing an expanded TIL population further comprising harvesting the expanded TIL population.

Once expanded, the disclosed expanded TIL population can be used for the treatment of cancer. In one aspect, disclosed herein are methods of treating a cancer in a subject comprising administering to the subject the rapidly expanded TIL of any preceding aspect. In other words, disclosed herein, in one aspect, are methods of treating cancer in a subject comprising obtaining a core biopsy (such as, for example, a core needle biopsy) comprising TIL from the subject; plating the core biopsy (such as, for example, a core needle biopsy) comprising TIL in a gas permeable reservoir; culturing the cells from the biopsy in a complete media comprising IL-2 for 5 weeks or less; harvesting the expanded TIL cells; and adoptively transferring to the subject the expanded TILs

The TILs that were rapidly expanded by the disclosed methods can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: sarcoma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

### 1. Pharmaceutical Carriers/Delivery of Pharmaceutical Products

As described above, the TILs can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 2. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include a needle for the removal of a core biopsy (such as, for example, a core needle biopsy), a core biopsy instrument, media to culture core biopsy tissue sample, IL-2, and/or anti-41BB discussed in certain embodiments of the methods, as well as the buffers and enzymes required.

### 3. Examples

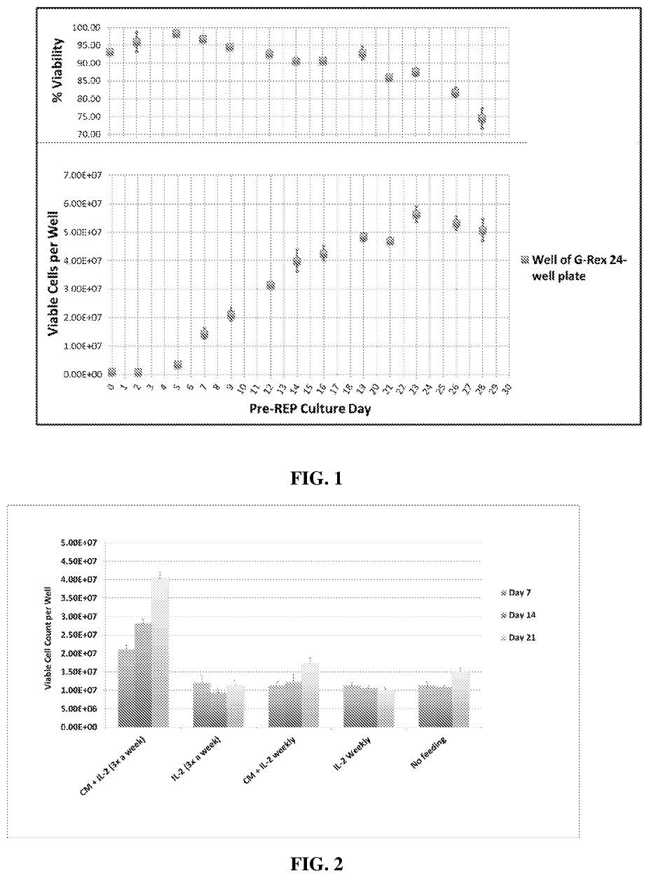

To explore the limitations of Pre-REP TIL culturing in the G-REX 24-well plates, the the maximum viable TIL count was tested in one well of the G-REX 24-well plates. Each well of a G-REX 24-well plate was seeded with 10⁶ Pre-REP TIL and added the maximum capacity (8 ml) of TIL complete media supplemented with 6000 IU IL-2/ml to each well. Fifty percent of the media was replenished 3 times a week. The TIL was cultured for 28 days. Every 3-4 days, two wells from each plate were harvested separately and cell count and viability were measured. As shown in FIG. 1, an average of 56×10⁶ (+/−3×10⁶) TIL were obtained per well of the G-REX 24-well plate before the cells reached the stationary growth phase. The TIL had an acceptable viability range of 90 to 98 percent before reaching the stationary phase

Since 56×10⁶ cells exceed our requirement for viable TIL count per cultured tumor fragment for REP culture initiation and since the viability was acceptably high, it was concluded that each tumor fragment could be cultured in one well of a G-REX 24-well plate without the need to split the TIL in multiple wells.

The optimal feeding schedule was then explored. Each of 45 wells of G-REX 24-well plates was seeded with 1×10⁶ Pre-REP TIL and added the maximum capacity (8 ml) of TIL complete media supplemented with 6000 IU IL-2/ml. To the first 9 wells, 50% of the media was replenished 3 times a week with TIL complete media and 6000 IU IL-2/ml. To the second 9 wells, only the IL-2 was replenished 3 times a week. To the thirds 9 wells, 50% of the media was replenished with TIL complete media and 6000 IU IL-2/ml only once a week. To the fourth 9 wells, only the IL-2 was replenished once a week. To the final 9 wells, neither the media nor IL-2 was replenished throughout the 21 day Pre-REP culture period. Three wells from each feeding group were harvested each week to measure viable cell count and viability. As indicated in FIG. 2, while the reduced feeding schedules did not significantly impact cell viability, they resulted in a halt in TIL growth at about 10×10⁶ cells per well. It was concluded that TIL in the G-REX 24-well plates grew optimally with 50% of TIL Complete media and 6000 IU/IL-2 two to three times a week.

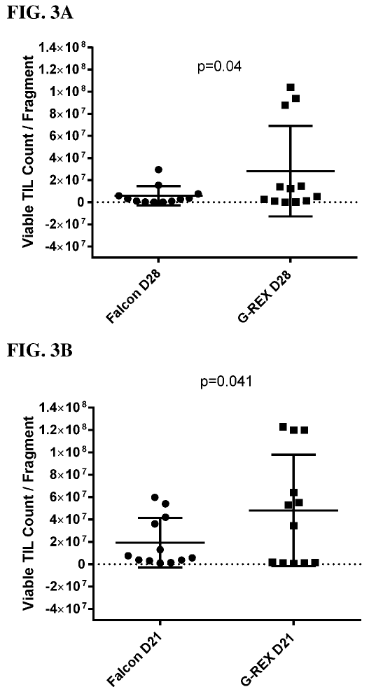

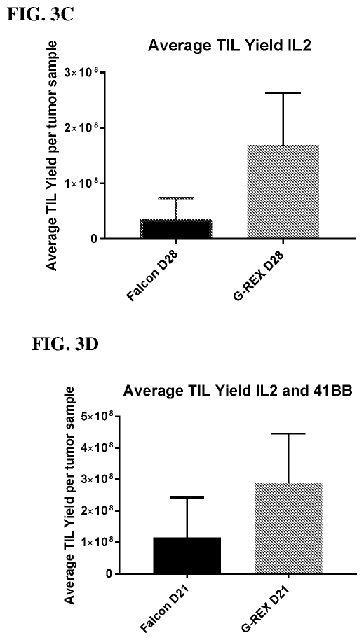

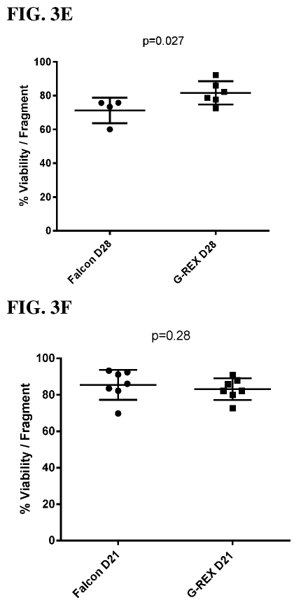

It was next investigated whether the new methodology would improve viability count, TIL yield, or percent yield after expansion relative to traditional methods and if any improvement would be increased through the addition of anti-4-1BB. Indeed, as shown in FIG. 3A, TIL culture from tumor fragments of two patients in G-REX 24-well plates resulted in a significant increase, 1-tailed t-test) in the average viable cell count per fragment compared to the conventional 24-well plates in the presence or absence of anti-41BB. FIG. 3B shows that TIL cultured from tumor fragments of two patients (6 fragments per patient) in G-REX 24-well plates resulted in a 3.8-fold increase in TIL average yield for cells that were cultured in IL-2 (1.7×10⁸ vs. 3.55×10⁷ total cells, n=2) and a 1.5-fold increase when cultured in presence of both IL-2 and anti-41BB (2.9×10⁸ vs. 1.16×10⁸, n=2). FIG. 3C shows the % viability of transferred the average percentage viability per expanded fragment (>6×10⁶ viable cells) was significantly higher when TIL was cultured from fragments in the presence of IL-2 for 28 days using the G-REX 24-well plates (81.63+/−6.9) compared to the conventional polystyrene 24-well culture plates (71.3%+/−7.5) (p=0.027, 1 tailed t-test, n=6, 4). There was no significant difference in the average percentage viability when TIL was cultured from fragments in the presence of IL-2 and anti-41BB for 21 days, where the percentage viability was high in both cases (G-REX=83.14, conventional=85.49, p=0.28, 1 tailed t-test, n=7, 7).

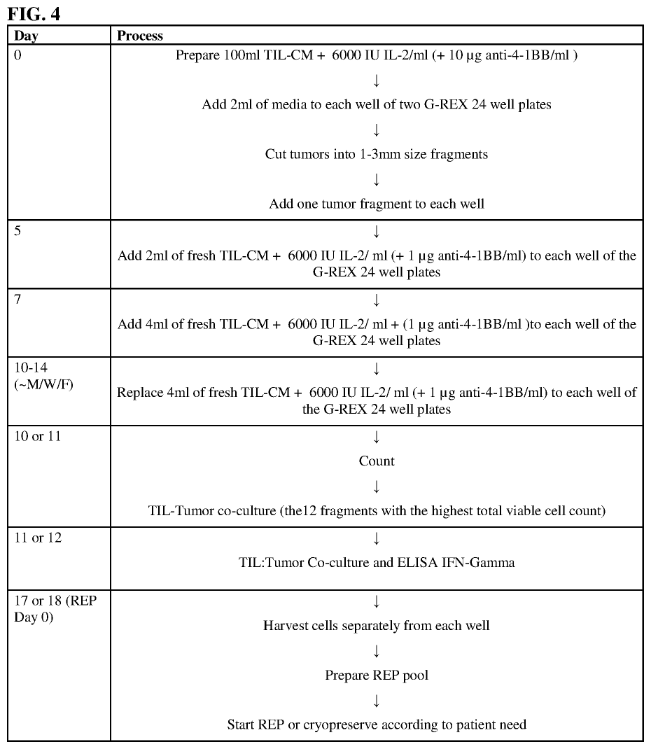

From this data a new culture protocol using GREX plates was devised. FIG. 4 shows the shorter Pre-REP new protocol: Viable TIL count/fragment TIL REP, typically a 2 weeks process is initiated after 24 to 38 days of TIL Pre-REP culture from fresh tumor fragments. Since we witnessed a substantial increase in the TIL count using the G-REX 24-well plates for Pre-REP, we decided to validate a plan where REP culture would start on day 16-18 We evaluated the shorter Pre-REP protocol using two more tumors (11,12 fragments each) in the presence of IL-2 and anti-41BB.

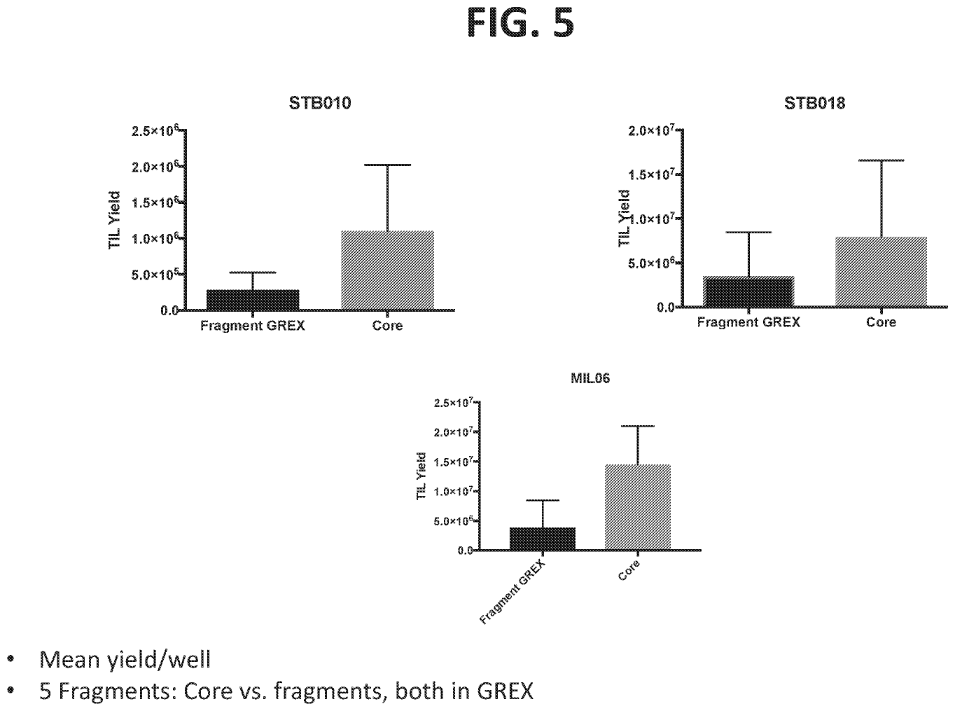

Next, it was investigated whether the improved method could accommodate core biopsies. The use of core biopsies would provide a significant advantage over traditional methods if the core biopsy could be used directly avoiding any cutting of a tumor into fragments, and/or processing said fragments. Moreover, the use of a single core per well would account for tumor heterogeneity. As shown in FIG. 5, comparing traditionally prepared fragments to core biopsy grown in a GREX24 well plate, the core biopsy showed significant increase in TIL yield. This also confirmed that the differences in yield in the new method (now using core biopsy) was not solely an artifact of GREX24 plates as the core biopsy provided a unexpected increase in yield.

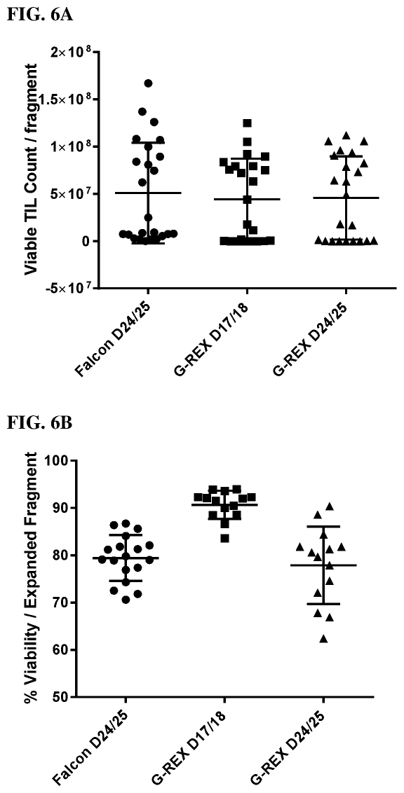

Next, the yield and viability of TILs produced by the new methodology was compared to traditional TIL production methods over time. FIG. 6A shows the TIL count per fragment. Sufficient TIL yield for rapid expansion was achieved using a single G-REX well per fragment (4.4×10⁷±4.3×10⁷, D17/18) a full seven days prior to a comparable yield from multiple polystyrene wells (5.1×10⁷±5.3×10⁷, D24/25) (p=0.32). Prolonged culture in the G-REX well did not significantly increase the number of TIL per fragment (4.6×10⁷±4.4×10⁷, D24/25) (p=0.45). FIG. 6B shows the % Viability per expanded fragment. TIL grown in G-REX wells showed higher viability (91±3%) on D17/18 compared to polystyrene on D24/25 (79±5%) (p<0.000001). The viability of TIL in G-REX wells decreased on D24/25 (78±8%) suggesting peak expansion occurred at the earlier time point. Thus, the revised core biopsy protocol produces sufficient TIL yield 7 days faster in GREX24 plates than traditional plates and was not improved by further culture (6A), but further culture to show a reduction in viability (6B).

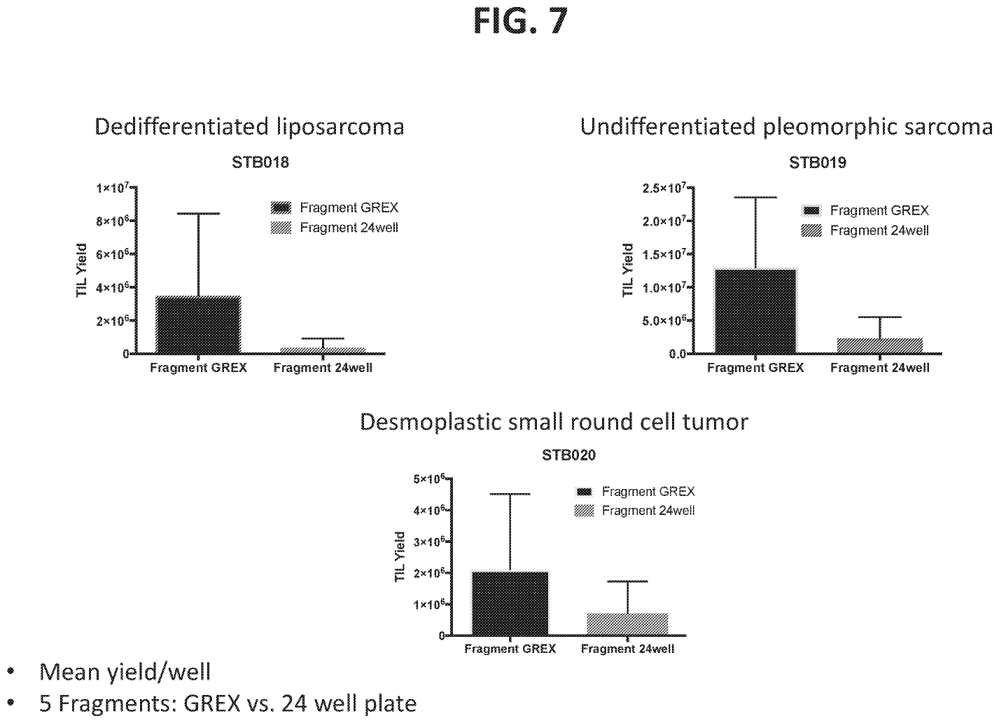

To determine both if the results observed were applicable to different tumor types, the methodologies were compared in dedifferentiated liposarcoma, undifferentiated pleomorphic sarcoma, and desmoplastic small round cell tumors. FIG. 7 shows that the increased viability in GREX24 plates using core biopsies and the new methodology compared to conventional methodology (i.e., surgical resection followed by fragment production, digest, and culture of multiple fragments in a single well of a regular 24-well plate) was maintained when culturing dedifferentiated liposarcoma, undifferentiated pleomorphic sarcoma, and desmoplastic small round cell tumors.

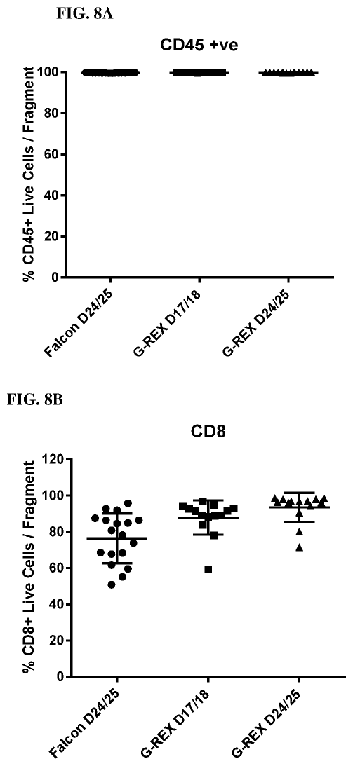

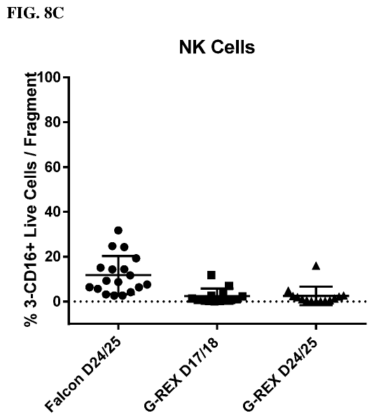

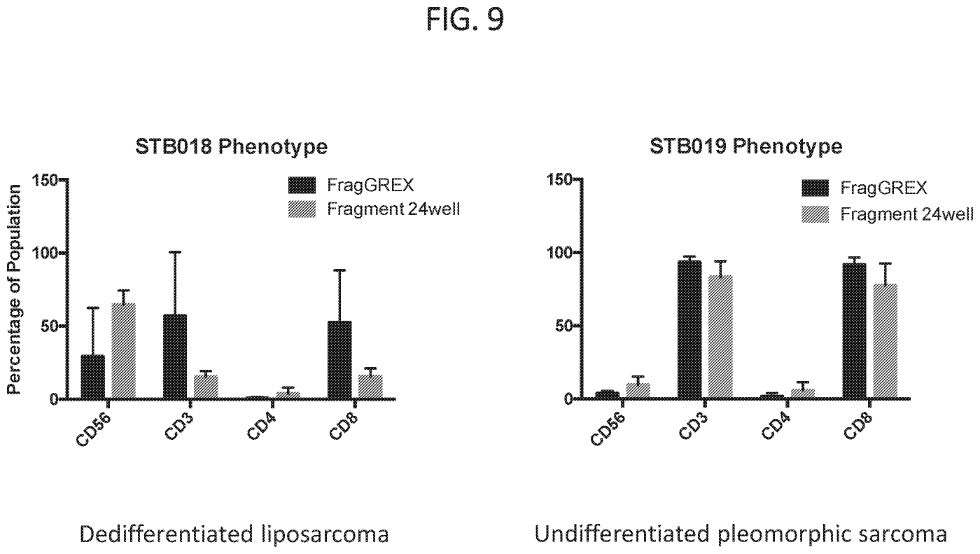

FIGS. 8A, 8B, and 8C show the Immune Phenotype of Shorter Pre-REP protocol. No tumor expansion as indicated by % CD45 (99.89%) when using the rapid TIL culture protocol in G-REX 24 well plates from tumor fragments (FIG. 8A). As shown in FIG. 8B, TIL cultured in G-REX have a higher percentage of CD8+ T cells (88±10%, D17/18; 94±8%, D24/25) than TILs from polystyrene (76±14%, D24/25 (p<0.001). However, as shown in FIG. 8C, TIL cultured in G-REX have a lower percentage of NK cells (G-REX: D17/18, 2.4±3%; D24/25, 3±4%; polystyrene: 12±9% (p<0.001). Looking at the phenotype of TILs in different tumor types, it was observed that the CD8 T cells, but not CD4 T cells increased significantly in all tumor types tested (FIG. 9).

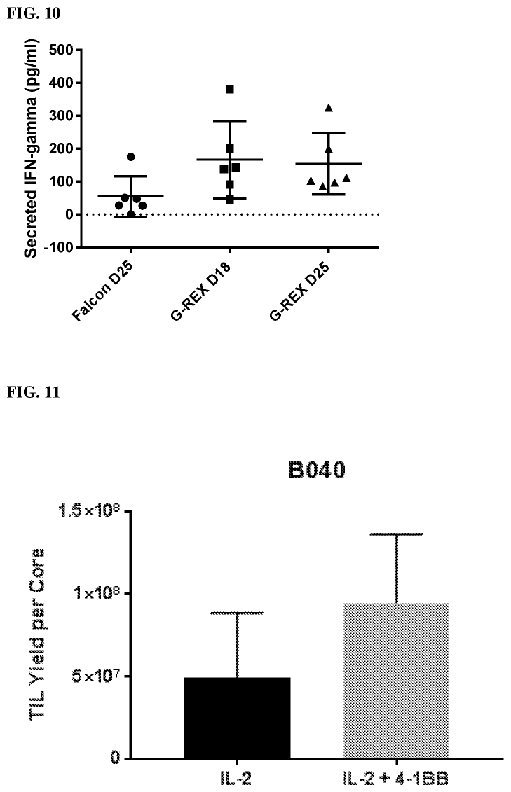

Next the reactivity against autologous tumor of Shorter Pre-REP protocol was investigated (FIG. 10). Significant increase in concentration of secreted IFN-g of the G-REX group at day 18 when co-cultured with autologous tumor (167±117 pg/ml) compared to the conventional polystyrene group (55±25 pg/ml, p=0.03) which may suggest increased TIL reactivity to autologous tumor when cultured in G-REX 24-well plates compared to conventional polystyrene plates. Prolonged culture in the G-REX well to 22 days did not affect the TIL function as indicated by the appearance of no significant decrease in the concentration of secreted IFN-g ((154±193.27 pg/ml, p=0.42).

Thus, the methods disclosed herein allowed us to obtain sufficient pre-REP TIL (60×10⁶ viable cells) for clinical dose manufacture in one week less than conventional polystyrene culture plates. Reducing the manufacturing period by one week potentially decreases patient ineligibility due to disease progression during TIL manufacturing. Moreover, the viability and % CD8+ cells were increased in GREX versus polystyrene tissue culture plates hile the % NK cells was reduced. In short, TIL culture in G-REX 24-well plates from tumor fragments is a simple and rapid method that may facilitate adoption of TIL therapy to other clinical sites.

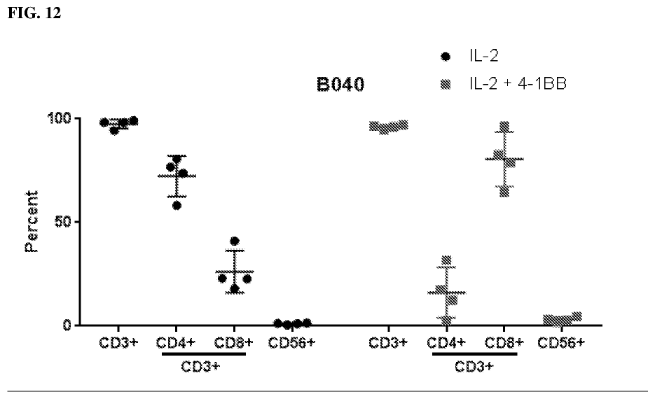

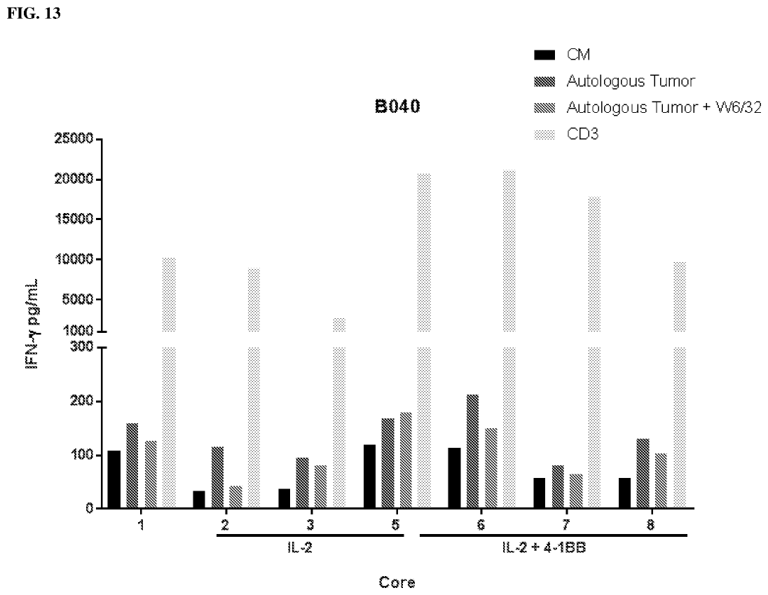

It was next investigated whether any improvement to the new methodology would be observed through the addition of anti-4-1BB. In particular whether the addition of anti-4-BB would increase yield, the percent of CD8 T cells, or result in higher anti-tumor activity (for example, IFN-γ release). Indeed, as shown in FIG. 11, TIL culture from tumor fragments of bladder cancer patients in G-REX 24-well plates resulted in a significant increase when co-cultured with IL-2 and anti-4-1BB resulting in an approximately 2-fold increase (5×10⁷ IL-2 alone vs 1×10⁸ IL-2+ anti-4-1BB). Moreover, as seen in FIG. 12, the addition of anti-4-1BB resulted in a drastic change in the ration of CD4:CD8 T cells with IL-2 alone having a 75:25 ratio of CD4:CD8 and IL-2+ anti-4-1BB resulting in a 25:75 ratio of CD4:CD8. Lastly, IFN-g secretion was measured and compared between bladder cancer cores from three subjects cultured with IL-2 alone or IL-2 and anti-4-1BB (FIG. 13). The co-culture of 11-2 and anti-4-1BB resulted in an average of a 50% increase in IFN-γ secretion relative to IL-2 alone.

What is claimed is:

1. A method of rapidly producing an expanded tumor infiltrating lymphocytes (TIL) population comprising:
a) directly plating TILs obtained from a tissue sample in a gas permeable reservoir;
b) culturing the TILs in media comprising IL-2;
c) exchanging the media in the reservoir at least 2 times per week.

2. The method of claim 1, wherein the TIL population is obtained from one or more core biopsy tissue samples.

3. The method of claim 2, wherein the one or more core biopsies are plated without disaggregating the specimen.

4. The method of claim 2, further comprising performing one or more core biopsies before plating the TILs.

5. The method of claim 2, wherein each reservoir contains a single intact core biopsy.

6. The method of claim 1, wherein the media is complete media.

7. The method of claim 6, wherein the complete media further comprises anti-41BB (anti-CD137) antibody.

8. The method of claim 1, wherein the gas permeable reservoir is a GREX24 gas permeable tissue culture multi-well plate.

9. The method of claim 1, further comprising harvesting the expanded TIL population.

10. The method of claim 1, wherein the TILs are cultured in media comprising IL-2 for 5 weeks or less.

11. A method of treating a cancer in a subject comprising administering to the subject the rapidly expanded TIL population of claim 1.

12. A method of treating cancer in a subject comprising obtaining one or more core biopsies comprising TILs from the subject; plating the one or more core biopsies comprising TILs each in a gas permeable reservoir; culturing the cells from the one or more biopsies in a complete media comprising IL-2 for 5 weeks or less; harvesting the expanded TIL cells; adoptively transferring to the subject the expanded TILs.
